(19) United States
(12) Patent Application Publication
(10) Pub. No.: US 2006/0058674 A1
(43) Pub. Date: Mar. 16, 2006

(54) Optimizing ultrasound acquisition based on ultrasound-located landmarks

(75) Inventor: Bjorn Olstad, Stathelle (NO)

Correspondence Address: MCANDREWS HELD & MALLOY, LTD, 500 WEST MADISON STREET, SUITE 3400, CHICAGO, IL, 60661, US

(73) Assignee: General Electric Company

(21) Appl. No.: 11/082,294

(22) Filed: Mar. 17, 2005

Related U.S. Application Data
(60) Provisional application No. 60/606,078, filed on Aug. 31, 2004.

(51) Int. Cl. A61B 8/02

(52) U.S. Cl. 600/450

(57) ABSTRACT

An ultrasound device is disclosed that includes a method and apparatus for generating an image responsive to moving cardiac structure and blood, and for adjusting at least one acquisition parameter based on, at least in part, locating at least one anatomical landmark within a heart. At least one processor, responsive to signals received from the heart, locates anatomical landmarks within the cardiac structure, generate position information of the anatomical landmarks, and adjusts at least one acquisition parameter. The landmarks and acquisition parameters may be displayed to a user of the ultrasound device.

### RELATED APPLICATIONS/INCORPORATION BY REFERENCE

This application is related to, and claims benefit of and priority from, Provisional Application No. 60/606,078 filed Aug. 31, 2004, titled “OPTIMIZING ULTRASOUND ACQUISITION BASED ON ULTRASOUND-LOCATED LANDMARKS”, the complete subject matter of which is incorporated herein by reference in its entirety.

The complete subject matter of each of the following U.S. patent applications is incorporated by reference herein in their entirety:
U.S. patent application Ser. No. 10/248,090 filed on Dec. 17, 2002.
U.S. patent application Ser. No. 10/064,032 filed on Jun. 4, 2002.
U.S. patent application Ser. No. 10/064,083 filed on Jun. 10, 2002.
U.S. patent application Ser. No. 10/064,033 filed on Jun. 4, 2002.
U.S. patent application Ser. No. 10/064,084 filed on Jun. 10, 2002.
U.S. patent application Ser. No. 10/064,085 filed on Jun. 10, 2002.

### FEDERALLY SPONSORED RESEARCH OR DEVELOPMENT

[Not Applicable]

### BACKGROUND OF THE INVENTION

Echocardiography is a branch of the ultrasound field that is currently a mixture of subjective image assessment and extraction of key quantitative parameters. Evaluation of cardiac wall function has been hampered by a lack of well-established parameters that may be used to increase the accuracy and objectivity in the assessment of, for example, coronary artery diseases. Stress echo is one example. It has been shown that the subjective part of wall motion scoring in stress echo is highly dependent on operator training and experience. It has also been shown that inter-observer variability between echo-centers is unacceptably high due to the subjective nature of the wall motion assessment.

Much technical and clinical research has focused on this problem, aimed at defining and validating quantitative parameters. Encouraging clinical validation studies have been reported, which indicate a set of new potential parameters that may be used to increase objectivity and accuracy in the diagnosis of, for instance, coronary artery diseases. Many of the new parameters have been difficult or impossible to assess directly by visual inspection of the ultrasound images generated in real-time. The quantification has typically required a post-processing step with tedious, manual analysis to extract the necessary parameters. Determination of the location of anatomical landmarks in the heart is no exception. Time intensive post-processing techniques or complex, computation-intensive real-time techniques are undesirable.

A method in U.S. Pat. No. 5,601,084 to Sheehan et al. describes imaging and three-dimensionally modeling portions of the heart using imaging data. A method in U.S. Pat. No. 6.099,471 to Torp et al. describes calculating and displaying strain velocity in real time. A method in U.S. Pat. No. 5,515,856 to Olstad et al. describes generating anatomical M-mode displays for investigations of living biological structures, such as heart function, during movement of the structure. A method in U.S. Pat. No. 6,019,724 to Gronningsaeter et al. describes generating quasi-realtime feedback for the purpose of guiding procedures by means of ultrasound imaging.

### BRIEF SUMMARY OF THE INVENTION

An embodiment of the present invention provides an ultrasound system for imaging a heart and automatically adjusting acquisition parameters after having automatically located anatomical landmarks within the heart. An apparatus is provided in an ultrasound machine for imaging a heart and adjusting certain acquisition parameters based on locating anatomical landmarks within the heart. In such an environment an apparatus for automatically adjusting acquisition parameters comprises a front-end arranged to transmit ultrasound waves into a structure and to generate received signals in response to ultrasound waves backscattered from said structure over a time period. A processor is responsive to the received signals to generate a set of analytic parameter values representing movement of the cardiac structure over the time period and analyzes elements of the set of analytic parameter values to automatically extract position information of the anatomical landmarks and track the positions of the landmarks. A processor is responsive to the tracked anatomical landmark positions and automatically adjusts certain acquisition parameters based on the tracked anatomical landmarks. A display is arranged to overlay indicia corresponding to the position information onto an image of the moving structure to indicate to an operator the position of the tracked anatomical landmarks, and to display the acquisition parameters, if desired.

A method is also provided in an ultrasound machine or device for imaging a heart and adjusting acquisition parameters based on having previously located certain anatomical landmarks within the heart. In such an environment, a method for automatically adjusting certain acquisition parameters comprises transmitting ultrasound waves into a structure and generating received signals in response to ultrasound waves backscattered from the structure over a time period. A set of analytic parameter values is generated in response to the received signals representing movement of the cardiac structure over the time period. Position information of the anatomical landmarks is automatically extracted and the positions of the landmarks are then tracked. Certain acquisition parameters are automatically adjusted based on the tracked anatomical landmarks. Indicia corresponding to the position information are overlaid onto the image of the moving structure to indicate to an operator the position of the tracked anatomical landmarks, and the adjusted acquisition parameters may also be displayed. Certain embodiments of the present invention relate to automatically adjusting at least one acquisition parameter (e.g., a depth setting, a width setting, an ROI position, a PRF setting, a gain setting, etc.) after automatically locating key anatomical landmarks of the heart, such as the apex and the AV-plane. In least one embodiment adjusting the at least one acquisition parameter is responsive to the at least one anatomical landmark and ultrasound data acquired in regions related to the anatomical landmarks.

### BRIEF DESCRIPTION OF THE DRAWINGS

FIG. 1 is a diagram of an embodiment of an ultrasound machine or device made in accordance with various aspects of the present invention.

FIG. 2 is a flowchart of an embodiment of a method performed by the machine or device shown in FIG. 1, in accordance with various aspects of the present invention.

FIG. 3 is a diagram illustrating using the method of FIG. 2 in the ultrasound machine of FIG. I to adjust acquisition parameters after having automatically located anatomical landmarks within the heart, in accordance with various embodiments of the present invention.

FIG 4 illustrates using the method of FIG. 2 to preset two longitudinal M-modes through two AV-plane locations in accordance with an embodiment of the present invention.

FIG. 5 illustrates using the method of FIG. 2 to preset a curved M-mode within a myocardial segment from the apex and down to the AV-plane in accordance with an embodiment of the present invention.

FIG. 6 illustrates using the method of FIG. 2 to preset a Doppler sample volume relative to detected anatomical landmarks in accordance with an embodiment of the present invention.

FIG. 7 illustrates using the method of FIG. 2 to define a set of points within myocardial segments to perform edge detection in accordance with an embodiment of the present invention.

The foregoing summary, as well as the following detailed description of certain embodiments of the present invention, will be better understood when read in conjunction with the appended drawings. It should be understood, however, that the present invention is not limited to the arrangements and instrumentality shown in the attached drawings.

### DETAILED DESCRIPTION OF THE INVENTION

An embodiment of the present invention enables the automatic adjusting of acquisition parameters, after locating and tracking certain anatomical landmarks of the heart, for subsequent acquisition of certain clinically relevant information. Moving cardiac structure and blood is monitored to accomplish the function. As used herein, structure means non-liquid and non-gas matter, such as cardiac wall tissue for example. An embodiment of the present invention helps establish improved, real-time visualization and assessment of wall function parameters of the heart. The moving structure is characterized by a set of analytic parameter values corresponding to anatomical points within a myocardial segment of the heart. The set of analytic parameter values may comprise, for example, tissue velocity values, time-integrated tissue velocity values, B-mode tissue intensity values, tissue strain rate values, blood flow values, and mitral valve inferred values.

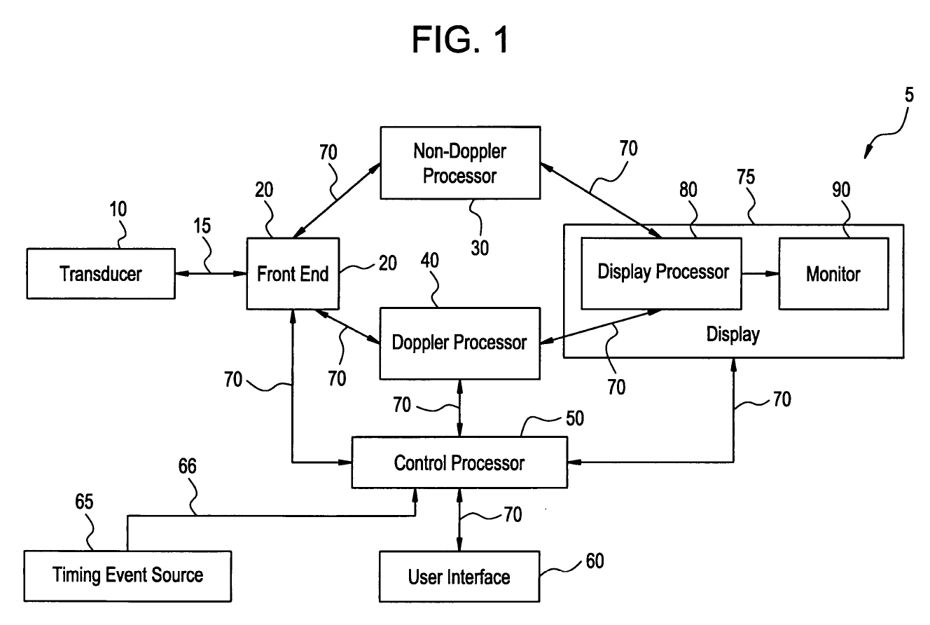

FIG. 1 depicts a diagram of an embodiment of an ultrasound machine 5 made in accordance with various aspects of the present invention. A transducer 10 is used to transmit ultrasound waves into a subject by converting electrical analog signals to ultrasonic energy and to receive ultrasound waves backscattered from the subject by converting ultrasonic energy to analog electrical signals. A front-end 20, comprising a receiver, transmitter, and beamformer, is used to create the necessary transmitted waveforms, beam patterns, receiver filtering techniques, and demodulation schemes that are used for the various imaging modes. Front-end 20 performs the functions by converting digital data to analog data and vice versa. Front-end 20 interfaces at an analog interface 15 to transducer 10 and interfaces over a digital bus 70 to a non-Doppler processor 30 and a Doppler processor 40 and a control processor 50. Digital bus 70 may comprise several digital sub-buses, each sub-bus having its own unique configuration and providing digital data interfaces to various parts of the ultrasound machine 5.

Non-Doppler processor 30 comprises amplitude detection functions and data compression functions used for imaging modes such as B-mode, B M-mode, and harmonic imaging. Doppler processor 40 comprises clutter filtering functions and movement parameter estimation functions used for imaging modes such as tissue velocity imaging (TVI), strain rate imaging (SRI), and color M-mode. The processors, 30 and 40, accept digital signal data from the front-end 20, process the digital signal data into estimated parameter values, and pass the estimated parameter values to processor 50 and a display 75 over digital bus 70. The estimated parameter values may be created using the received signals in frequency bands centered at the fundamental, harmonics, or sub-harmonics of the transmitted signals in a manner known to those skilled in the art.

Display 75 comprises scan-conversion functions, color mapping functions, and tissue/flow arbitration functions, performed by a display processor 80 which accepts digital parameter values from processors 30, 40, and 50, processes, maps, and formats the digital data for display, converts the digital display data to analog display signals, and passes the analog display signals to a monitor 90. Monitor 90 accepts the analog display signals from display processor 80 and displays the resultant image to the operator on monitor 90.

A user interface 60 allows user commands to be input by the operator to the ultrasound machine 5 through control processor 50. User interface 60 comprises a keyboard, mouse, switches, knobs, buttons, track ball, and on screen menus.

A timing event source 65 is used to generate a cardiac timing event signal 66 that represents the cardiac waveform of the subject. The timing event signal 66 is input to ultrasound machine 5 through control processor 50.

Control processor 50 is in at least one embodiment, the central processor of the ultrasound machine 5 and interfaces to various other parts of the ultrasound machine 5 through digital bus 70. Control processor 50 executes the various data algorithms and functions for the various imaging and diagnostic modes. Digital data and commands may be transmitted and received between control processor 50 and other various parts of the ultrasound machine 5. As an alternative, the functions performed by control processor 50 may be performed by multiple processors, or may be integrated into processors 30, 40, or 80, or any combination thereof. As a further alternative, the functions of processors 30, 40, 50, and 80 may be integrated into a single PC backend.

Once certain anatomical landmarks of the heart are identified, (e.g., the AV-planes and apex as described in U.S. patent application Ser. No. 10/248,090 filed on Dec. 17, 2002) certain acquisition parameters may be automatically adjusted by the ultrasound system 5 in order to optimize subsequent acquisition of clinically relevant information, in accordance with various aspects of the present invention. The various processors of the ultrasound machine 5 described above may be used to adjust and position the various acquisition parameters.

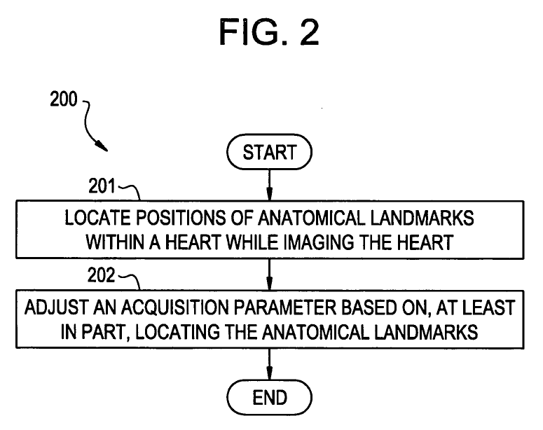

FIG. 2 depicts a flow chart of an embodiment of a method 200 performed by the machine 5 of FIG. 1 in accordance with various aspects of the present invention. In step 201, positions of anatomical landmarks (e.g., the AV-plane and apex) are identified or located within the heart while imaging the heart. In step 202, an acquisition parameter is automatically adjusted based on, at least in part, having located and identified the positions of the anatomical landmarks. In least one embodiment adjusting the at least one acquisition parameter is responsive to the at least one anatomical landmark and ultrasound data acquired in regions related to the anatomical landmarks.

As defined herein, acquisition parameters include, for example, at a depth setting of an ultrasound image, a width setting of an ultrasound image, a position of a region-of-interest (ROI), a pulse-repetition-frequency (PRF) setting of the ultrasound machine, a gain setting of the ultrasound machine, an adaptive gain setting of the ultrasound machine, at least one transmit focus position of the ultrasound machine, and a position of a 3D acquisition region or some combination.

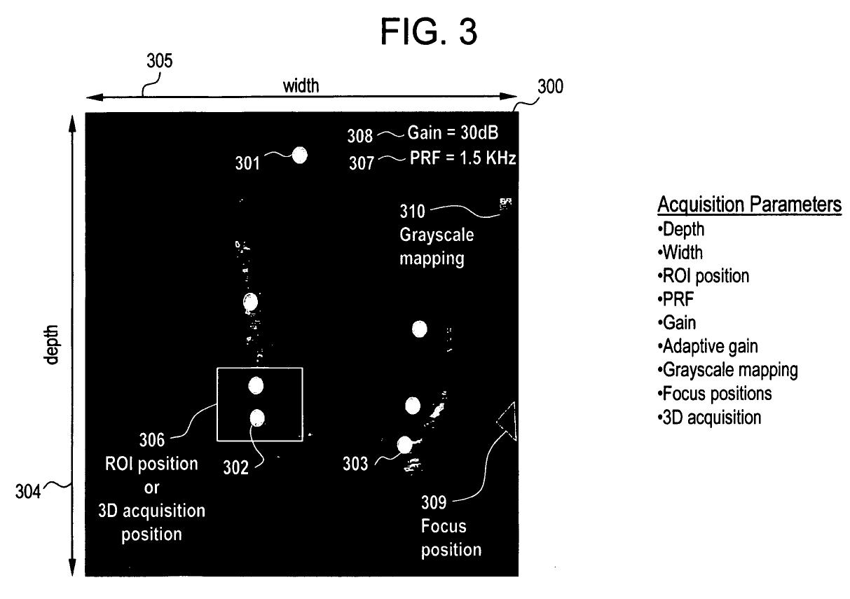

FIG. 3 is a diagram illustrating using the method 200 of FIG. 2 in the ultrasound machine 5 of FIG. 1 to adjust acquisition parameters after having automatically located anatomical landmarks within the heart, in accordance with various embodiments of the present invention. FIG. 3 shows a displayed B-mode image 300 of a heart also displaying the locations of anatomical landmarks of the heart and various acquisition parameters. The displayed anatomical landmarks include an apex location 301, a first AV-plane location 302, and a second AV-plane location 303. The displayed acquisition parameters include a depth setting 304 for the B-mode image 300, a width setting 305 for the B-mode image 300, a positioned region-of-interest (ROI) or 3D acquisition region 306, a pulse-repetition-frequency (PRF) setting 307 of the ultrasound machine 5, a gain setting 308 of the ultrasound machine 5, a positioned transmit focus 309 of the ultrasound machine 5, and a grayscale mapping 310 for the B-mode image 300.

For example, the depth 304 and/or width 305 may be adjusted by the ultrasound machine 5 based on the locations of the apex 301 and the AV-planes 302 and 303 to yield an appropriate B-mode view relative to actual heart dimensions. For example, the depth 304 and width 305 settings may be adjusted such that the displayed B-mode image of the heart is actual size. The appropriate depth and width settings are calculated from the relative positions of the apex and AV-plane locations.

As another example, after detecting, the AV-plane 302 for example, a color flow ROI 306 may be automatically positioned over the AV-plane 302 to visualize mitral flow near the AV-plane location. Alternatively, as part of locating the AV-plane 302, for example, the highest tissue velocities in the region around the AV-plane 302 may be determined and used to automatically adjust the PRF setting 307 such that the highest velocity may be resolved in, for example, a tissue velocity imaging (TVI) ROI 306.

As a further example, a gain setting, adaptive gain setting, and/or gray scale mapping may be adjusted to obtain a known transfer function at identified anatomical locations within the image 300. Such an approach serves to standardize the grayscale mapping and is beneficial for the visual appearance of the image and for the reduction of variance in subsequent automated procedures that may be performed such as, for example, edge detection.

The position of a transmit focus 309 may be automatically adjusted to, for example, follow the position (in depth) of the AV-plane 303. As a result, a best lateral resolution of the AV-plane 303 may be maintained. Alternatively, multiple transmit focus positions may be adjusted over the depth of the image 300 between, for example, the apex 301 and the AV-plane 302 in order to maximize image quality between the two.

One of the primary applications of cardiac 3D is that of rendering heart valves. The identification of AV-plane locations may be used to improve a 3D acquisition of a heart valve by positioning an acquisition ROI (e.g., 306) in a more optimal location.

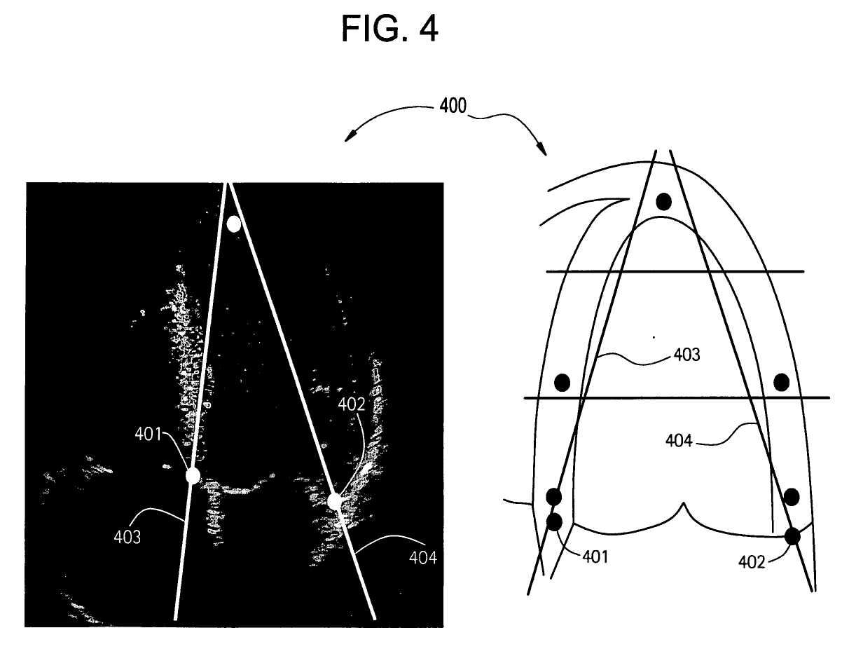

FIG. 4 depicts a diagram that illustrates using method 200 of FIG. 2 to preset two longitudinal M-modes through two AV-plane locations, extracting information, in accordance with an embodiment of the present invention. FIG. 4 illustrates how two longitudinal M-modes 403 and 404 may be preset through the two AV-plane locations 401 and 402 in order to display the longitudinal AV-motion in two M-modes within the heart 400, in accordance with an embodiment of the present invention.

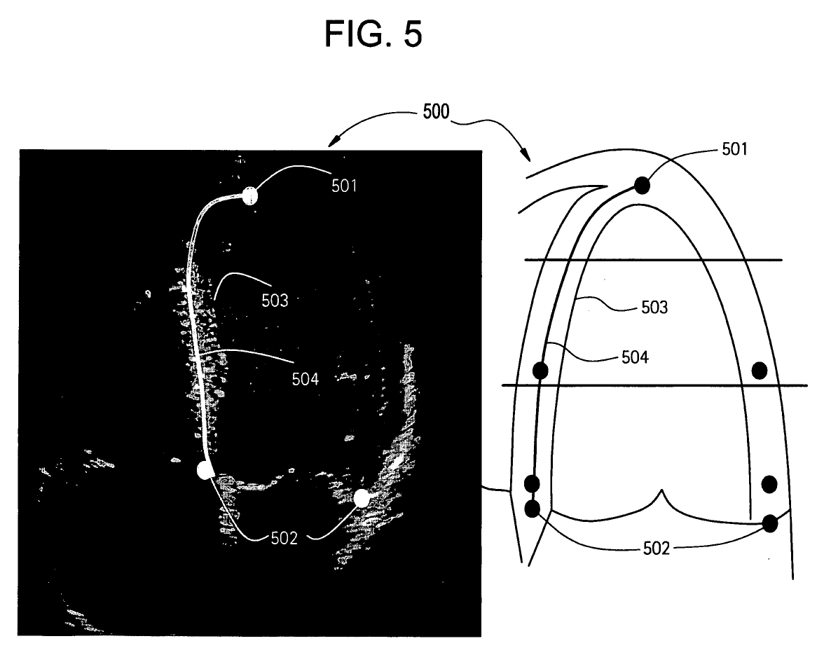

FIG. 5 depicts a diagram illustrating using method 200 of FIG. 2 to preset a curved M-mode within a myocardial segment from apex down to the AV-plane, extracting information, in accordance with an embodiment of the present invention. FIG. 5 illustrates how a curved M-mode 504 from apex 501 down to the AV-plane 502 in the middle of myocardium 503 may be preset using the landmarks alone or in combination with local image analysis to keep the curve 504 inside myocardium 503 within the heart 500, in accordance with an embodiment of the present invention.

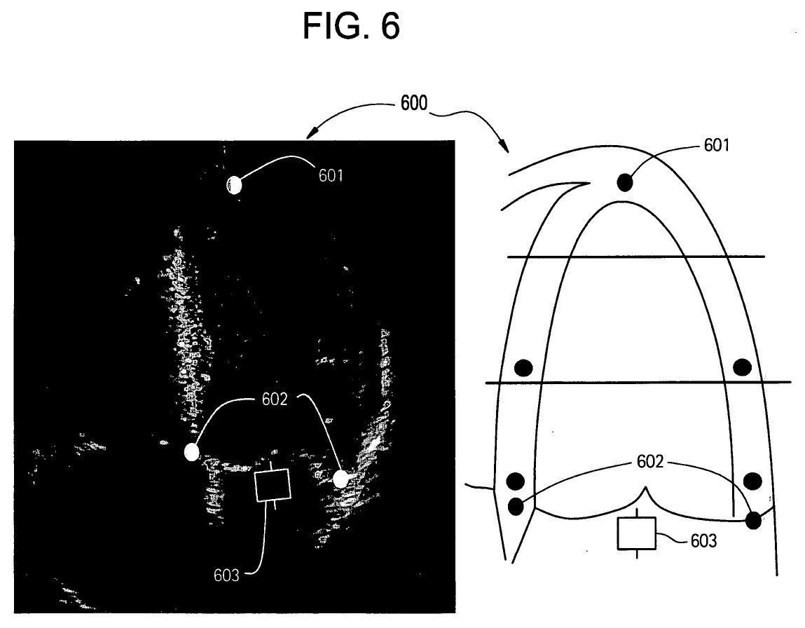

FIG. 6 depicts a diagram illustrating using methods 200 of FIG. 2 to preset a Doppler sample volume relative to detected anatomical landmarks, extracting information, in accordance with an embodiment of the present invention. FIG. 6 illustrates how a sample volume 603 for Doppler measurements may be preset relative to the detected landmarks 601 (apex) and 602 (AV-plane) within the heart 600. Such a technique may be applied to PW and CW Doppler, for inspection of blood flow and measurement of myocardial function.

In accordance with at least one embodiment of the present invention, a region-of-interest (ROI) may be preset with respect to the anatomical landmarks extracting information from these clinically relevant locations. The extracted information may include one or more of Doppler information over time, velocity information over time, strain rate information over time, strain information over time, M-mode information, deformation information, displacement information, and B-mode information.

The locations of the M-modes, curved M-modes, sample volumes, and ROI's may be tracked in order to follow the motion of the locations, in accordance with an embodiment of the present invention. Further, indicia may be overlaid onto the anatomical landmarks and/or the clinically relevant locations to clearly display the positions of the landmarks and/or locations.

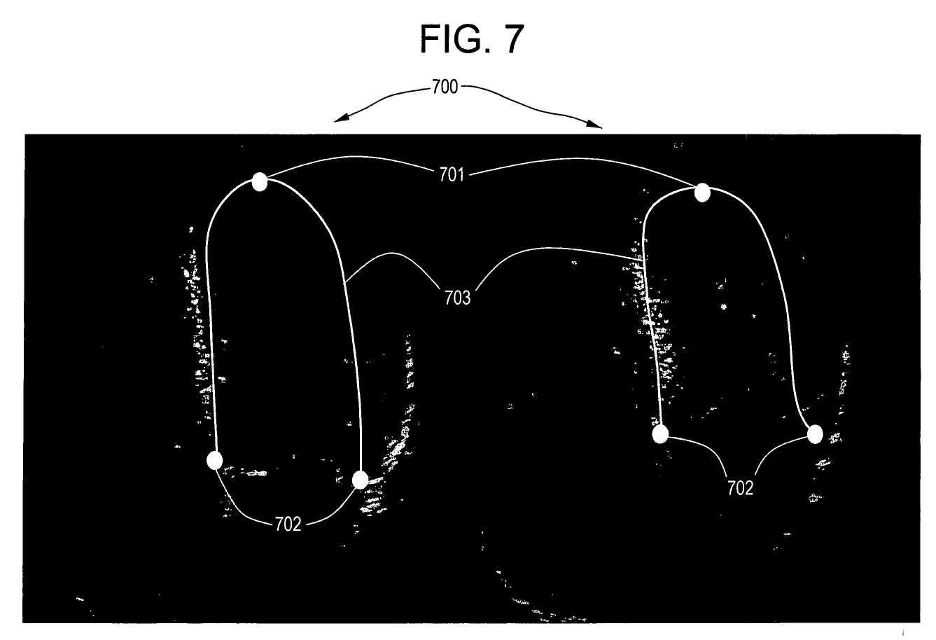

FIG. 7 depicts a diagram illustrating using method 200 of FIG. 2 to define a set of points within myocardial segments performing edge detection to extract information about the associated endocardium, in accordance with an embodiment of the present invention. Automatic edge detection of the endocardium remains a challenging task. FIG. 7 illustrates how the techniques discussed herein (i.e., similar to the curved M-mode localization) may be used to either define a good ROI for the edge detection, or provide an initial estimate that may be used to search for the actual boundary with edge detection algorithms such as active contours. FIG. 7 illustrates two views of a heart 700 identifying the apex 701 and the AV-plane 702. A contour 703, estimating the approximate inside of myocardial segments in the heart 700 based on the anatomical landmarks, is drawn as the apex and AV-plane locations are tracked. Edge detection of the endocardium may then be performed using edge detection techniques using the contour as a set of starting points.

In accordance with an alternative embodiment of the present invention, other locations within a heart such as, for example, lower parts of mid segments and basal segments, may also be identified and used to adjust certain acquisition parameters.

While the invention has been described with reference to certain embodiments, it will be understood by those skilled in the art that various changes may be made and equivalents may be substituted without departing from the scope of the invention. In addition, many modifications may be made to adapt a particular situation or material to the teachings of the invention without departing from its scope. Therefore, it is intended that the invention not be limited to the particular embodiment disclosed, but that the invention will include all embodiments falling within the scope of the appended claims.

1. In an ultrasound device for generating an image responsive to moving cardiac structure and blood within a heart of a subject, a method comprising:
locating at least one anatomical landmark within the cardiac structure and generating position information of said at least one anatomical landmark; and
adjusting at least one acquisition parameter based on, at least in part, locating said at least one anatomical landmark.

2. The method of claim 1 wherein adjusting said at least one acquisition parameter is responsive to said at least one anatomical landmark and ultrasound data acquired in regions related to said anatomical landmarks.

3. The method of claim 1 wherein said at least one anatomical landmark comprises at least one of an apex of said heart and an AV-plane of said heart.

4. The method of claim 1 wherein said acquisition parameter comprises a depth setting of an ultrasound image.

5. The method of claim 1 wherein said acquisition parameter comprises a width setting of an ultrasound image.

6. The method of claim 1 wherein said acquisition parameter comprises a position of a region-of-interest (ROD.

7. The method of claim 1 wherein said acquisition parameter comprises a pulse-repetition-frequency (PRF) setting of the ultrasound device.

8. The method of claim 1 wherein said acquisition parameter comprises a gain setting of the ultrasound device.

9. The method of claim 1 wherein said acquisition parameter comprises an adaptive gain setting of the ultrasound device.

10. The method of claim 1 wherein said acquisition parameter comprises a grayscale mapping of said ultrasound machine.

11. The method of claim 1 wherein said acquisition parameter comprises at least one transmit focus position of the ultrasound device.

12. The method of claim 1 wherein said acquisition parameter comprises a position of a 3D acquisition region.

13. In an ultrasound device for generating an image responsive to moving cardiac structure and blood within a heart of a subject, an apparatus comprising:
a front-end arranged to transmit ultrasound waves into said moving cardiac structure and blood and to generate received signals in response to ultrasound waves backscattered from said moving cardiac structure and blood;
at least one processor responsive to said received signals to locate at least one anatomical landmark within said cardiac structure and generate position information of said at least one anatomical landmark and adjust at least one acquisition parameter based on, at least in part, locating said at least one anatomical landmark.

14. The method of claim 12 wherein said at least one processor is responsive to said at least one anatomical landmark and ultrasound data acquired in regions related to said anatomical landmarks when adjusting said at least one acquisition parameter.

15. The apparatus of claim 13 further comprising a display processor and monitor to process said position information and display indicia overlaying at least one of said at least one anatomical landmark.

16. The apparatus of claim 13 further comprising a display processor and monitor to process and display said at least one acquisition parameter.

17. The apparatus of claim 13 wherein said at least one anatomical landmark comprises at least one of an apex of said heart and an AV-plane of said heart.

18. The apparatus of claim 13 wherein said at least one acquisition parameter comprises at least one of a depth setting of an ultrasound image, a width setting of an ultrasound image, a position of a region-of-interest (ROI), a pulse-repetition-frequency (PRF) setting of the ultrasound device, a gain setting of the ultrasound device, an adaptive gain setting of the ultrasound device, at least one transmit focus position of the ultrasound device, a grayscale mapping of said ultrasound image, and a position of a 3D acquisition region.

19. The apparatus of claim 13 wherein said at least one processor comprises at least one of a Doppler processor, a non-Doppler processor, a control processor, and a PC back-end.

20. The apparatus of claim 13 further comprising at least one transducer connected to said front-end to convert electrical signals to said ultrasound waves.

21. The apparatus of claim 13 comprising at least one transducer connected to said front-end to convert said ultrasound signals to electrical signals.

22. The apparatus of claim 13 further comprising at least one user interface connecting to said at least one processor to control operation of the ultrasound device.
